(19) United States
(12) Patent Application Publication
(10) Pub. No.: US 2003/0177512 A1
(43) Pub. Date: Sep. 18, 2003

(54) Method of genetically altering and producing allergy free cats

(76) Inventor: David B. Avner, Charlottesville, VA (US)

Correspondence Address: FOLEY AND LARDNER, SUITE 500, 3000 K STREET NW, WASHINGTON, DC, 20007, US

(21) Appl. No.: 10/295,903

(22) Filed: Nov. 18, 2002

Related U.S. Application Data
(63) Continuation of application No. 09/227,873, filed on Jan. 11, 1999, now abandoned.
Application No. 09/227,873 is a continuation-in-part of application No. 08/657,905, filed on Jun. 7, 1996, now abandoned.
(60) Provisional application No. 60/000,189, filed on Jun. 13, 1995.

(51) Int. Cl. A01K 67/027; C07H 21/04; C12P 21/02; C12N 5/06

(52) U.S. Cl. 800/14; 536/23.2; 435/325

(57) ABSTRACT

A transgenic cat with a phenotype characterized by the substantial absence of the major cat allergen, Fel d I. The phenotype is conferred in the transgenic cat by disrupting the coding sequence of the target gene with a specialized construct. The phenotype of the transgenic cat is transmissible to its offspring.

### CROSS-REFERENCE TO RELATED PATENT APPLICATIONS

This application is a continuation of U.S. application Ser. No. 09/227,873, filed on Jan. 11, 1999, which in a continuation-in-part of U.S. application Ser. No. 08/657,905, filed on Jun. 7, 1996, which claims priority to provisional U.S. application Ser. No. 60/000,189, filed Jun. 13, 1995, each incorporated in its entirety by reference.

### FIELD OF THE INVENTION

This invention relates to the production of transgenic animals wherein a recognized gene sequence, coding for an identified allergen, is inactivated. More particularly, the invention relates to transgenic cats wherein the gene sequences, coding for the major cat allergen Fel d I, have been disrupted.

### BACKGROUND OF THE INVENTION

Approximately 6 million Americans are allergic to cats, and although many persons allergic to cats do not have cats in their own homes, almost one third do. It has been suggested that 28% of homes in the United States have at least one cat (which equals at least 50 million cats). Patients allergic to cats often report a rapid onset of asthma and rhinitis upon entering a house with a cat. When tested, almost all of these patients will show a positive immediate hypersensitivity skin test to extracts of cat dander and will have serum IgE antibodies against cat allergens (Luczynska, JACI, August 1989).

To date, most treatments to cat sensitivity have centered around avoidance and immuno-therapy. Avoidance can mean considerable alterations in ones living environment and daily routines. For example, in order to avoid excessive exposure to indoor allergens it is recommended that carpets be removed from floors, bedding be covered with special sheets, air conditioners be cleaned regularly, and air be filtered with costly air filters. The time, effort and expense often makes this type of treatment unappealing to allergy sufferers.

Immunization can be an effective treatment for allergies. Unfortunately, the expense of regular allergy shots, the time involved to receive treatment, and the variability of effectiveness are considerable deterrents for some patients. Furthermore, there is risk that a patient may have a severe reaction to the immunization and can even go into anaphylactic shock.

### SUMMARY OF THE INVENTION

This invention is a new alternative to traditional treatments for allergies. Rather than recommending avoidance or immuno-therapy, this invention eliminates the allergen at its source. In the case of the cat, sensitivity has been attributed to one major cat allergen (Fel d I) (Ohman, JACI, 1977). Using, newly developed gene targeting techniques it is possible to “knock-out” the Fel d I genes in an embryonic cell ie. Embryonic Stem (ES) Cells. These modified ES cells can then be introduced into a developing blastomere. During normal embryonic development the ES cells will then be incorporated into part of the germ line (Capecchi, Science, June 1989), (Robbins, Circulation Research, July 1993).

The resulting chimeric offspring will be heterozygous for the inactive Fel d I gene. When cross-bred with another heterozygous cat, one fourth of the progeny will be homozygous to the inactive Fel d I gene. These homozygous cats are major allergen free and are a revolutionary alternative to immuno-therapy for allergic cat owners (FIG. 1).

This invention is applicable to all animals in which a specific allergen can be identified and in which the disruption of the gene sequence coding for the particular allergen causes no harm to the animal.

This invention is based on the production of transgenic animals in which the gene sequence for a particularly allergen has been disrupted by a specialized construct rendering the gene inactive. In the preferred embodiment the altered gene will be transmissible to the offspring.

Embryonic stem cells are pluripotent cells derived from the inner cell mass of the blastocyst. These cells retain the ability to differentiate into any tissue type in the developing body. A change in the genomic sequence of an ES cell will be passed on to all other cells derived directly from the altered ES cell line.

The Fel d I gene coding for the major cat allergen is disrupted or “knocked-out” in the embryonic stem cell of a cat. This is accomplished by inserting into or replacing part of the functional gene with a new sequence of genomic DNA, rendering the gene inactive. The modified ES cell can then be introduced into a developing blastomere by one of several recognized techniques and then implanted into a pseudopregnant foster cat. During normal embryonic development, cells derived from the altered ES cell are incorporated in part of the germ line and somatic tissue.

The resulting chimeric offspring are heterozygous for the inactivated Fel d I gene. When cross-bred with another heterozygous cat, approximately one fourth of the progeny will be homozygous for the inactive Fel d I gene. These cats are major cat allergen free. The altered gene and subsequent phenotype is transmissible to future offspring.

The invention provides an isolated polynucleotide sequence encoding a disrupted Fel d I gene. In accordance with the invention, such a sequence can be disrupted by sequence replacement, sequence insertion, or deletion of all or a part of said Fel d I gene. In further embodiments of the invention, a nucleotide sequence encoding a selectable marker is inserted into the Fel d I gene or used to replace all or part of the Fel d I gene. An example of such a selectable marker gene is a gene that confers neomycin resistance.

In another embodiment of the invention, there is provided a recombinant polynucleotide vector comprising all or part of a disrupted Fel d I gene. In yet another aspect of the invention, there is provided an embryonic cat stem cell comprising a disrupted Fel d I gene and an embryonic cat stem cell comprising a vector which in turn comprises a disrupted Fel d I gene.

In yet another embodiment, the present invention provides a transgenic cat comprising a disrupted Fel d I gene. The Fel d I gene of the somatic cells, the germ line cells, or both the somatic and germ line cells of such a transgenic cat may be disrupted. In accordance with the invention, there is provided a transgenic cat which is heterozygous for the disrupted Fel d I allergen gene. There also is provided a transgenic cat which is homozygous for said disrupted Fel d I gene. Transgenic cats comprising a disrupted Fel d I gene are provided that are fertile and capable of transmitting said disrupted Fel d I gene to its offspring are also provided.

The present invention also provides a first method for producing a transgenic cat comprising a disrupted Fel d I gene, comprising the steps of:

(a) introducing a cat stem cell comprising a disrupted Fel d I gene into a cat embryo;

(b) transplanting said embryo into a pseudopregnant cat; and

(c) allowing said cat embryo to mature into a cat.

Transgenic cats produced in accordance with this method can be heterozygous or homozygous for the disrupted Fel d I gene. Homozygous transgenic cats will not produce the Fel d I cat allergen.

Finally, in another embodiment of the present invention, there is provided a second method for producing a transgenic cat comprising a disrupted Fel d I gene, wherein said cat does not produce the cat allergen Fel d I, and wherein said cat is homozygous for said disrupted Fel d I gene, comprising the steps of:

(a) producing a first heterozygous transgenic cat according to the first method described above;

(b) producing a second heterozygous transgenic cat according to the first method described above, wherein said second cat is not the same sex as said first cat;

(c) breeding said first and second cats; and

(d) selecting transgenic cats which are homozygous for said disrupted Fel d I gene and do not produce Fel d I antigen.

### BRIEF DESCRIPTION OF THE DRAWINGS

FIG. 1 is a schematic summary of the generation of cat germ line chimeras from embryo-derived stem cells containing a targeted gene disruption.

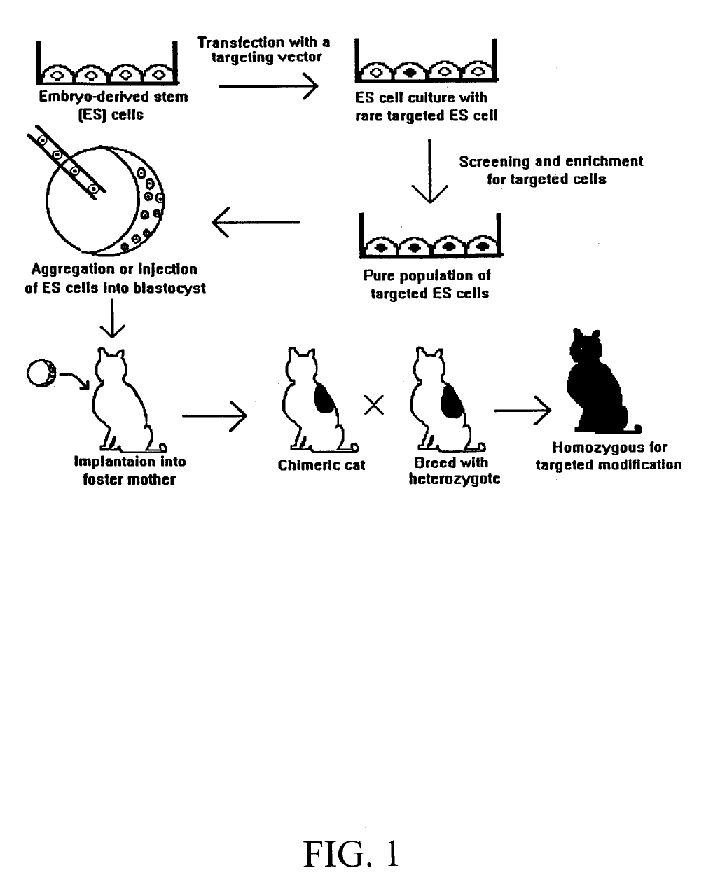

FIG. 2 shows the nucleotide sequence of chain 1 (Ch 1) of the Fel d I gene in a cat. Ch 1 is composed of a mature protein subunit of 70 aa. Sequencing of the gene encoding for Ch 1 demonstrates that there are two alternative Ch 1 leader sequences with the leader B exon separated from the start on the leader A exon by an intron of 46 bp. The junction of leader B (exon 1) or leader A (exon 2) with exon 3 leads to alternative codons that encode either Asp (leader B) or Asn (leader A). These junctions (exon 1/3 and exon 2/3) are positioned 2 aa from the N terminus of the mature Ch 1, which starts with Glu¹. The structural gene is comprised of only two exons, 3 and 4, that encode the mature protein.

FIG. 3 shows the nucleotide sequence of chain 2 (Ch 2) of the Fel d I gene in a cat. Ch 2 is composed of a mature protein subunit of 92 aa. The leader sequence and the first 3 aa of the mature protein are encoded by exon 1 (61 nucleotides (nt): 20 aa). The bulk of the mature protein is encoded by exons 2 and 3 (aa 4-64 and 65-90, respectively). The first 18 nt of exon 3 of Griffith's published sequence encode the residues, IAINEY (aa 65-70)(Expression and Genomic Structure of the Genes Encoding FdI, the Major Allergen from the Domestic Cat, Gene (1992)), rather than Morgenstern's published sequence, TTISSSKD, suggesting that Ch 2 has two forms (Morgenstern, et al., Proc. Nat'l. Acad. Sci. USA, 88:9690 (1991)).

FIG. 4. depicts a schematic for a sequence replacement vector. Sequence replacement vectors are designed such that upon linearization, the vector sequences remain collinear with the endogenous sequences. Following homologous pairing between vector and genomic sequences, a recombination event replaces the genomic sequences with the vector sequences containing the neo^{r} gene. A strp^{s} gene can be place outside of the homologous coding region of the replacement vector to make future screening of ES cell colonies easier. Open boxes indicate introns; closed boxes indicate exons; the crosshatched box indicates the neo^{r} gene.

FIG. 5. depicts a schematic for a sequence insertion vector. Sequence insertion vectors are designed such that the ends of the linearized vector lie adjacent to one another on the Fel d I map. Pairing of these vectors with their genomic homolog, followed by recombination at the double strand break, results in the entire vector being inserted into the endogenous gene. This produces a duplication of a portion of the Fel d I gene. Open boxes indicate introns; closed boxes indicate exons; the crosshatched box indicates the neo^{r} gene.

FIG. 6. depicts the construction of the neo^{r} gene. The structural gene and its control elements are contained on a 1 kb cassette flanked by an Xhol site (x) and a Sall site (s) in a pUC derivative plasmid. (a) A tandem repeat of the enhancer region from the polyoma mutant PYF441 consisting of bases 5210-5274. (b) The promoter of HSV-tk, from bases 92-218. (c) A synthetic translation initiation sequence, GCCAATATGGGATCGGCC. (d) The neo^{r} structural gene from Tn5, including bases 1555-2347.

### DETAILED DESCRIPTION OF THE INVENTION

I. Transgenics

While this disclosure pertains to transgenic cats it is not limited to said species. The invention herein pertains to all animals in which a gene coding for an allergenic protein can be identified and inactivated without causing harm to the animal. The term “animal” is used herein to include all vertebrate animals, except humans. It also includes an individual animal in all stages of development, including embryonic and fetal stages. A transgenic “animal” is any animal containing one or more cells bearing genetic information received, directly or indirectly, by deliberate genetic manipulation at a subcellular level, such as by microinjection, infection with recombinant virus, or electroporation. The genetic manipulation may be directed directly at the chromosome or it may be directed towards extrachromosomally replicating DNA. A “transgenic animal” refers to an animal in which the genetic information was introduced into a germ line cell, thereby conferring the ability to transfer the information to offspring. If such offspring in fact possess some or all of that information then they, too, are transgenic animals.

The following is presented by way of example and is not to be construed as a limitation on the scope of the invention.

II. The Embryonic Stem Cell

The key to the production of allergy free cats is the successful incorporation of new DNA into the ES cell. The generation of chimeras between embryonic stem (ES) cell lines or clones and embryos is an essential step in these processes, which when successful leads to the derivation of new strains of cats with an altered genome.

Most ES lines that are currently in use have an XY or male genotype. This has two advantages. The first is that male XY ES lines, when injected into female XX blastocysts, will tend to bias the development of the resulting chimera toward a male phenotype. In phenotypically male chimeras, only XY-bearing germ cells (i.e., those derived from the ES cells) will form functional gametes. XX primordial germ cells (i.e., those derived from the host blastocyst) will not form functional gametes and are lost. This will, therefore, favor the development of gametes derived from the ES cells. Second, a male chimera can produce more offspring over its reproductive life span than a female, so that even chimeras with a relatively low percentage contribution of the ES cells to the germ line can be detected.

The length of time that ES cells have spent in culture since their derivation can also affect their ability to make germ line chimeras. Chimeras that are the strongest and of the highest frequency are usually those derived with early passage clones (i.e., up to 10-15 passages); thereafter, it has been noted that the extent and frequency of chimerism may often, but not always, start to decline.

To generate germ line chimeras efficiently it is essential that the ES line be tested, prior to any manipulation or selection, for its capability of generating chimeras at a high frequency. The criterion is that more than 50% of the offspring born should be chimeric, with the majority of these being able to transmit the ES genotype through the germ line. It is also recommended to determine the karyotype of any subsequent clones isolated by selection, prior to injection into blastocysts, thereby avoiding any clones having aneuploid karyotypes that may not produce germ line chimeras. This procedure will result in considerable savings in time and effort and need only involve counting of the chromosomes, using the C -banding staining technique if the ES cell line used has already been assessed as to its ability to produce germ line chimeras. Any deviation from a mean number of chromosomes will almost inevitably result in weak chimeras being produced, with little possibility of the ES cells contributing to the germ line. The exception, however, is loss of the Y chromosome from a male ES line. Such clones can produce very good chimeras, resulting in germ line transmission by the females.

A. Derivation of Embryonic Stem Cells

The following procedures were adapted from the protocol described in Abbondanzo, Gadi, and Stewart, “Derivation of Embryonic Stem Cell Lines.” Methods in Enzymology, 1993. Embryonic stem (ES) cells are the pluripotent derivatives of the inner cell mass (ICM) of the blastocyst. ES cells are derived directly from the ICM of blastocysts explanted in vitro. A variety of procedures have been employed to obtain ES cells, including using blastocysts that have undergone delayed implantation as well as culturing cells directly from ICMs isolated from blastocysts following immunosurgery. The derivation of embryonic stem cells is disclosed in full in Abbondanzo, Gadi, and Stewart, Derivation of Embryonic Stem Cell Lines, Methods in Enzymology (1993).

The in vitro growth of ES cells is dependent on the cytokine leukemia inhibitory factor (LIF). This protein is essential for maintaining the growth of ES cells in vitro since, in its absence, ES cells differentiate and eventually will cease to proliferate.

Leukemia inhibitory factor can be supplied to ES cells in different ways. Currently the best approach, and still the most effective one for long-term culture, is to grow the ES cells on a feeder layer of fibroblasts. The feeder layers synthesize and secrete LIF into the culture medium, and, in addition, an alternative form of LIF is also produced that remains closely associated with the extracellular matrix deposited by the fibroblasts. LIF is the only factor produced by the feeder layers that is essential for ES cell growth.

Embryonic stem cell lines can also be established and maintained from embryos in the absence of a feeder layer. Under these conditions the culture medium is supplemented with recombinant LIF, which is available from commercial suppliers (GIBCO-BRL, Grand Island, N.Y.; R and D Systems, Minneapolis, Minn.). It is also possible to use regular culture medium supplemented with medium “conditioned” by growing certain cell lines (see below) that secrete relatively large quantities of LIF into the culture medium. The medium can be collected and used at an appropriate dilution as a source of LIF.

B. Culture Requirements

To establish and culture ES cells, a laboratory equipped with standard tissue culture facilities is required, namely, a sterile/filtered air culture hood, a 37CO₂-gassed incubator, and a tissue-culture microscope equipped with phase-contrast optics for viewing cells. In addition, a good stereo dissection microscope is required with ×40 magnification, along with a mouth-controlled pipette that is used for transferring blastocysts and for picking the ICMs or ES colonies. See (Abbondanzo et al., Methods in Enzymology, 1993)

C. Culture Media

The effective maintenance of ES cells requires that all culture media be made with very pure water. The Millipore (Bedford, Mass.) Five-bowl Milli-Q purification system provides water that is of satisfactory quality. A variety of different media have been used to culture embryos and ES cells: Dulbecco's modified Eagle's medium (DMEM), Glasgow modified Eagle's medium, and a DMEM/Ham's F12 mixture. Good results are obtained with DMEM with high glucose (4.5 g/liter), L-glutamine, and no sodium pyruvate. The me-dium is purchased in powdered form, although 1× to 10× concentrated liquid forms are available. It is made up according to the manufacturer's instructions and buffered with 2.2 g/liter sodium bicarbonate. It is supplemented with MEM nonessential amino acids to a final concentration of 0.1 mM [these can be obtained from GIBCO-BRL as a 100× (10 mM) solution]. In addition, L-glutamine to a final concentration of 2 mM is added together with 2-mercaptoethanol at a final concentration of 0.1 mM [a stock 0.1 M solution is made by adding 70 ul of the standard 14 M solution (Sigma, St. Louis, Mo.) to 10 ml of phosphate-buffered saline (PBS)]. Penicillin (50 IU/ml) and streptomycin (50 IU/ml) are also included in the final formulation, and 100× solutions can be obtained from GIBCO-BRL. This formulation is referred to as ES-DMEM. See (Abbondanzo et al., Methods in Enzymology, 1993)

D. Preparation of Feeder Layers

Embryonic stem cells are dependent on the cytokine LIF to maintain them as an undifferentiated proliferating population. The cytokine is usually supplied by growing the cells on mitotically inactive feeder layers of G418^{r} fibroblasts that produce LIF. (Ramirez-Solis et al., Methods in Enzymology, 1993), (Robbins, Circulation Research, 1993). Recombinant LIF is commercially available but is expensive. ES cells have been derived from blastocyst cultures in the absence of feeders, but with the medium supplemented with recombinant LIF. However, the majority of these lines contain a significant percentage of aneuploid karyotypes, rendering them unsuitable for the generation of germ line chimeras. Only in a few instances have germ line chimeras been produced with ES cells established in feeder-free LIF-containing medium. As yet it is unclear as to whether feeders are providing, in addition to LIF, other factors that help to establish and maintain ES cells. Possibly, the matrix-associated form of LIF, along with the extracellular matrix deposited by the feeders, is more effective in maintaining ES cells than the soluble form alone. It has been found that the maintenance of feeder-dependent ES cells, under feeder-free conditions in the presence of LIF, is more effective (in inhibiting ES differentiation) when the ES cells are grown on extracellular matrix deposited by fibroblasts rather than on gelatine alone, which is the standard procedure. See also (Abbondanzo et al., Methods in Enzymology, 1993)

The feeders can be permanently growing lines (e.g., STO fibroblasts). The advantage of STO cells is that they are continuously proliferating, so they do not need to be repeatedly derived. The disadvantage with STO cells is that there is variation between different sublines, with some being more effective than others at sustaining ES cells. The following procedure, described in Ramirez-Solis et al., Methods in Enzymology, 1993, can be used:

1. Coat tissue culture plates with gelatin (Gelatin solution: 1% (w/v) tissue culture grade gelatin mixed in water and sterilized by autoclaving; the working solution is 0.1% and is made by diluting the 1% stock solution in sterile water. Store at room temperature) by covering the bottom of the plate with a 0.1% gelatin solution and incubating at room temperature for 2 hr. Aspirate the gelatin before plating the inactivated feeder cells.

Grow G418^{r} cells to confluence on 15 cm gelatinized tissue culture plates in DMEM plus 7% FCS and 1× GPS. To inactivate the cells, mitomycin C stock solution (0.5 mg/ml) is added to the medium to give a final concentration of 10 ug/ml, and the plate is incubated at 37°, 5% (v/v) CO₂, for 2 hr.

4. Aspirate the mitomycin-containing medium and wash the plate twice with PBS.

4. Add 2 ml of trypsin solution and incubate at 37°, 5% CO₂, for 5 min.

5. Add 5 ml of medium and suspend the cells by vigorous pipetting. Transfer the cells to a 50-ml sterile centrifuge tube. Wash the plate with medium once again. Pool all the mitomycinr-treated cells and centrifuge at 1000 rpm for 5 min at room temperature.

6. Aspirate the supernatant and resuspend the pellet, in 5-10 ml of medium. Count the cells and add medium to give a concentration of 3.5×10⁵ cells/ml.

7. Transfer aliquots of feeders onto gelatinized plates, 12 ml per 10-cm plate (4.2×10⁶ cells/plate), 4 ml per 6-cm plate (1.4×10⁶ cells/ plate), etc. Leave plates in the incubator overnight before use to give cells time to attach to the plate. Feeder plates can be stored for 3-4 weeks in the incubator, but they should be checked under the microscope before use to confirm that the layer is intact.

E. Isolation of Embryonic Stem Cells from Blastocysts

The following procedure, described in Verstegen, Journal of Reproduction and Fertility (1993), can be used:

1. The experimental cats are housed under a lighting schedule of 14 h light and 10 h dark. The cats are fed once daily and allowed access to water ad libitum. Cats are examined daily to ensure that they are not in oestrus or close to the next oestrus period. Allow a 2 week separation between the beginning of the treatment and the end of the previous oestrus period.

2. pFSH without LH activity is reconstituted in physiological saline to a concentration of 2 iu/ml (1 iu=10 ug). Solutions can be aliquoted and stored at −20⁰° C. until use.

3. Inject each cat subcutaneously with 2.0 iu of pFSH daily for five days (each cat receives a total of 10.0 iu of pFSH).

4. On day six inject 1.0 iu of pFSH subcutaneously and 250.0 iu of human chorionic gonadotrophin (hCG) intramuscularly. Repeat these injections on the seventh day.

5. On Days 5,6,7, and 8, queens are placed with a fertile male until a minimum of four matings have occurred.

6. The surgical recovery of embryos are performed by uterine lavage between day 11 and day 13 after onset of treatment. The animals are anaesthetized with 100 ug medetomidine/kg and 5 mg ketamine/kg by intramuscular injection.

7. After a midline incision, the ovaries, the uterotubal junction and the body of the uterus are exteriorized.

8. Make a 1.0 mm incision in the uterine body and insert a three-way Swan-Ganz paediatric catheter into one uterine horn. Inflate the cuff to seal the distal end of the horn. At the uterotubal junction, an atraumatic needle is introduced in the uterine lumen and 20 ml of phosphate-buffered saline (PBS) [without Ca and Mg, plus pyruvate-Na (0.36 g/l), kanamycin sulfate (0.25 g/l) and phenol red (0.05 g/l)] warmed to 39⁰ C is injected twice into the horn. The flushing liquid is recovered via the Swan-Ganz catheter into an aseptic bottle.

9. After recovery, suture the incisions with 5/0 vicryl.

10. Transfer the embryos into a 35-mm culture dish containing PBS with 10% fetal calf serum (PBS-FCS).

The following additional steps, described by Abbondanzo et al., Methods in Enzymology, 1993, are also carried out:

11. Locate the embryos using a stereo dissection microscope with ×20 or ×40magnification. Once an embryo/blastocyst is identified, it is removed from the dish using a mouth-controlled pipette.

12. Transfer the embryos to a fresh dish of PBS-FCS to wash away any contaminating blood cells or uterine tissue and discard any unfertilized eggs/embryos.

13. The blastocysts are transferred to 60-mm dishes containing pre-pared feeders, adding no more than 20 to each dish. The ES-DMEM medium is supplemented with 1000 IU of recombinant LIF (murine or human is equally effective). The dishes with the embryos are returned to a 37⁰ incubator and left undisturbed for 2 days.

14. Over this period, embryos will hatch from the zona pellucida and attach to the surface of the dish. The trophoblast spreads out to form a monolayer of cells on which the inner cell mass (ICM) can be seen. Over the next 2 days (i.e., up to day 4 from the time of explanting the blastocysts), the ICM grows and forms a distinct mound of cells on the trophoblast monolayer. At the end of 4 days and in the first half of the fifth day of culture, the ICMs should be picked for disaggregation. There appears to be an optimal window in time when the ICM is best suited for producing ES lines. Generally, blastocysts are too far developed if picked any period after 5 days of explanting, and the frequency of forming ES lines declines. This point can often be recognized by the formation of an endoderm layer around the core of ICM. These explants rarely, if ever, give rise to ES lines.

To pick the ICMs, the culture medium is aspirated and the dish washed twice in Ca²⁺/Mg²⁺-free PBS, with embryos remaining covered by the PBS. Microdrops of 0.25% trypsin and 1.0 mM EDTA plus 1% chicken serum are set up under paraffin oil. Chicken serum is included in the trypsin-EDTA solution because, unlike FCS, it does not contain a trypsin inhibitor, and the added protein protects the cells from lysis.

The ICMs are picked off the trophoblast by gently dislodging them using a mouth-controlled pipette. Each ICM is then transferred into a single microdrop of trypsin-EDTA solution plus 1% chicken serum and left for approximately 3-5 min. The cells in the ICM clump start to lose contact with each other. Using another mouth-controlled pipette, whose tip has been flame-polished to remove any sharp edges and whose diameter is between 50 and 100 um, the clumps are broken up into smaller clusters of cells and single cells by pipetting up and down a few times. The entire cell suspension is transferred to a single well of a 16-mm tissue culture dish which already contains a fibroblast feeder layer. The culture medium (1 ml) is ES-DMEM supplemented with 1000 IU of LIF. Use Nunclone 4×16 mm well multidishes (Nunc) as the culture vessel for the disaggregated ICMs, allowing one well per ICM. When all the ICMs have been disaggregated and each one has been transferred to a well, the culture dishes are returned to the incubator.

Between 3 and 4 days after explanting the ICMs, the wells should be inspected to check that ICM cells are present and have started to form colonies. The explanted ICM cells do not just give rise to ES cells. In many instances, other cell types appear with the continued culture of the primary explants. These colonies may at first resemble ES colonies. However, over time they differentiate and cease to proliferate. ES cell colonies, which have a characteristic morphology continue to proliferate, usually as tight round colonies that have smooth edges. It is difficult to distinguish the individual cells in the colony, although their nuclei can be recognized and contain one or two prominent nucleoli. By observing the well on a daily basis, it is possible to see whether a colony continues to increase in size as it proliferates without differentiation. These colonies are most often found at the perimeter of the well, which is sometimes difficult to view with a tissue culture microscope. Careful inspection should therefore be made of the perimeter to ensure that no colonies are missed. ES colonies should be apparent within 7-10 days after picking and disaggregating the ICM.

It appears that using early passage (P2-3) fibroblasts and including recombinant LIF in the culture medium can help in the establishment of ES cells from the disaggregated ICMs. Overall, ES lines can be established at a frequency of 10-30% from the picked ICMs.

F. Expansion of Embryonic Stem Cells

When colonies of ES cells have been identified in the primary explants, their numbers can be expanded. It is not necessary to isolate the ES cells in the primary cultures from other differentiated cell types that may be present, since one of the characteristics of ES cells is rapid and continuous proliferation.

The entire well containing the ES colonies is washed 2 times in PBS, and the PBS is aspirated. To each well, 0.2 ml of trypsin solution plus 1% chick serum is added, and the well is left to trypsinize for 5 min. Then 0.5 ml of ES-DMEM is added, and all clumps of cells are broken up by gently pipetting the suspension, with care being taken to ensure that no bubbles are introduced into the well. If only one or two ES colonies are present in the well, the cell suspension is left in the well to reattach. The medium is replaced, the next day, with 1 ml of ES-DMEM plus 1000 IU/ml LIF. Over the next 3-5 days, if ES colonies were correctly identified, many new colonies of ES cells should become visible. The well can then be trypsinized again and the contents transferred to a 60-mm dish containing a fibroblast feeder layer. The colonies of ES cells should continue to proliferate without differentiation. At this point, it is no longer necessary to include LIF and the cells can be maintained on feeder layers in ES-DMEM. See procedure described in Abbondanzo, supra.

G. Expansion, Freezing, and Routine Culture of Embryonic Stem Cells

Once an ES line has been found to contain a high percentage of cells with a normal diploid karyotype, it should be expanded so that as many early passage cells as possible are frozen in liquid nitrogen. This will provide sufficient resources for future experiments, since early passage ES cells tend to make better chimeras at a higher frequency than if passages 15-20 and later are used. However, there is no absolute correlation, since relatively late passage lines such as D3 have been reported to produce germ line chimeras.

The ES cells can be maintained as an undifferentiated population by trypsinizing and replating the cells onto dishes containing fresh feeders, every 5-6 days if the cells are plated out at a sufficiently low density. A 60-mm dish at maximum density will contain about 1-2×10⁷ ES cells, and a 150-mm dish can contain up to 2-3×10⁸ cells at maximal density. The cells will start to differentiate or die if they are maintained beyond the maximum density level, and thus the optimal period of time they can be maintained before they have to be passaged is about 5-7 days. To maintain a line, trypsinizing a semiconfluent dish and plating out of the single cell suspension with 1:100 to 1:500 dilution is sufficient. If the cells are replated at reasonably low density, the culture medium needs changing every other day to keep cells under optimal conditions. If more cells and higher densities are required, then the cells should be refed every day. Under optimal conditions, the ES cells should grow as small clusters or mounds. If the conditions are suboptimal, differentiated derivatives will appear, and the mounds of ES cells will start to flatten out, with individual cells becoming more distinct. Under extreme conditions the majority of the cells will have differentiated. For a general description of this technique, see Abbondanzo, supra.

H. Freezing of Embryonic Stem Cells

The following technique, described by Abbondanzo, supra, can be used.

1. A culture of ES cells should be in the log phase of growth, that is, not at maximal density. Wash the dish 2 times in PBS and trypsinize.

2. Harvest the cells, resuspended in medium, and count with a hemocytometer.

3. The medium for freezing the cells consists of a 50:50 mixture of DMEM and FCS containing a final concentration of 10% (v/v) dimethyl sulfoxide (DMSO) (Sigma).

4. One milliliter of medium containing 1-5×10⁶ ES cells is aliquoted into a 1-ml sterile freezing vial (Nunc) that has a screw cap and rubber seal.

5. The vials are labeled with the ES line and passage number, placed in a holding rack, and left overnight in a −70° freezer.

6. The following day the frozen vials should be transferred to a liquid nitrogen container for long-term storage.

7. To thaw ES cells, a 60-mm tissue culture dish containing a feeder layer in ES-DMEM medium should be prepared in advance. Remove the vial of ES cells and place in a beaker of sterile distilled water prewarmed to 37⁰ until the contents of the vial have melted. Remove the vial, swab with 100% ethanol to sterilize the outside, and remove the cell suspension with a sterile Pasteur pipette. The cells can be immediately plated out in the 6-mm dish. The next day the culture medium is replaced with fresh ES-DMEM to remove all the DMSO and any dead cells. If freezing and thawing of the ES cells were performed correctly, then ES colonies should already be visible in the culture dish.

III. Gene Targeting

A. Culture of Embryonic Stem Cells

The following procedure is adapted from the protocol described in Ramirez-Solis, Davis, and Bradley, “Gene Targeting in Embryonic Stem Cells.” Methods in Enzymology, 1993).

The purpose of using ES cells for gene targeting is to transfer the mutation generated in culture into the cat germ line. For this reason, culture conditions that prevent the overgrowth of abnormal cells are critical. ES cells should be grown on mitotically inactivated feeder cell layers. In addition, the cells should be grown at high density and passaged frequently at 1:3 to 1:6; this usually means replacing the medium daily. ES cells should be fed 4 hr before passage. To passage, the cells should be washed twice with PBS and trypsinized for 10 min; there is no need to prewarm the trypsin solution. ES-DMEM medium is added, and the cell clumps are mechanically disrupted by vigorous pipetting. It is important to generate a single cell suspension before passage as clumps have a tendency to differentiate. The passage number of the cell line should be recorded to give an estimate of the time the cells have been in culture. If the cells are not to be used immediately, they should be frozen and then recovered when needed.

The cultured ES cell population includes totipotent cells, as well as cells with limited potential to contribute to all tissues of the cat. Be-cause targeted events are usually rare and single cell cloning is necessary, it is advisable to optimize targeting vectors and conditions such that several targeted clones can be recovered. Also, cloning involves culture at low cell concentrations and potentially for a prolonged period while screening for the desired clone.

B. Genes Encoding Fel d I

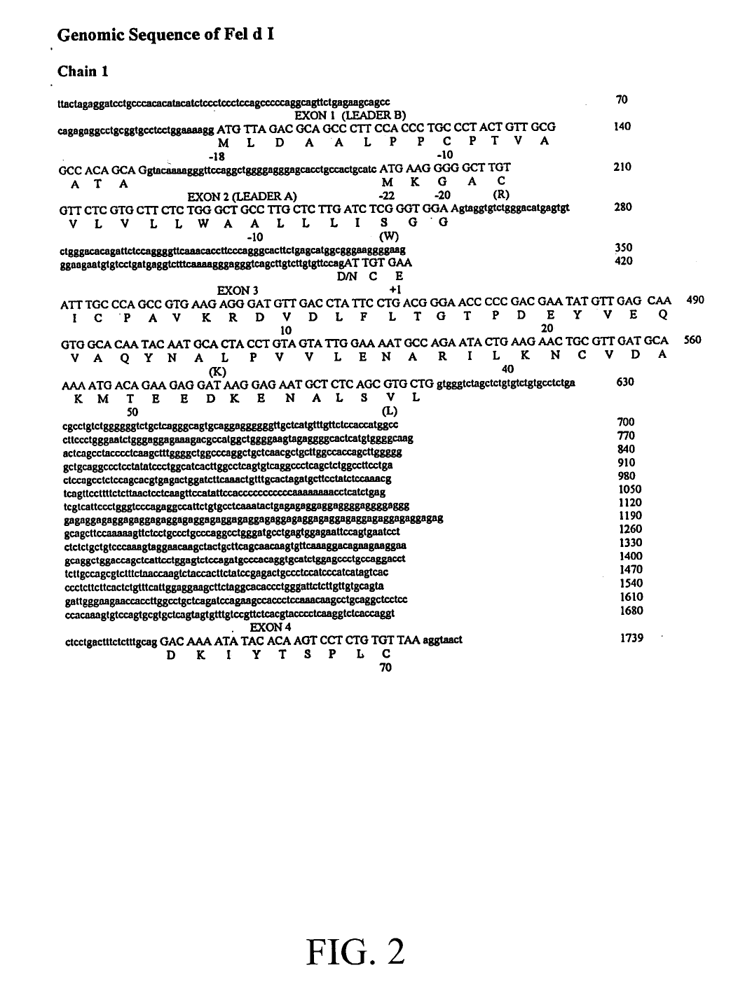

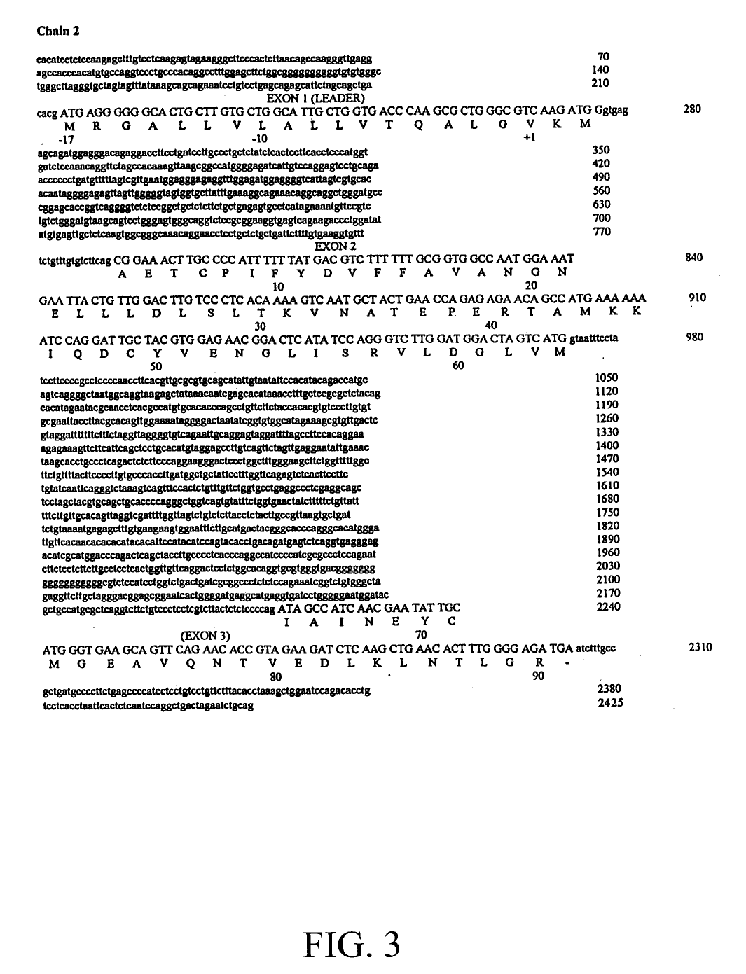

Two genes encode for the protein chains that comprise the major cat allergen, Fel d I. The protein chains are designated Ch 1 and Ch 2. One published polynucleotide sequence for the Fel d I gene is described in Griffith, et al. Expression and Genomic Structure of the Genes Encoding FdI, the Major Allergen from the Domestic Cat, Gene (1992), which is shown in FIGS. 2 and 3. See also Morgenstern, et al., Proc. Nat'l. Acad. Sci. USA, 88:9690 (1991).

Ch 1 is composed of a mature protein subunit of 70 aa. Sequencing of the gene encoding for Ch 1 demonstrates that there are two alternative Ch 1 leader sequences with the leader B exon separated from the start of the leader A exon by an intron of 46 bp. The junction of leader B (exon 1) or leader A (exon 2) with exon 3 leads to alternative codons that encode either Asp (leader B) or Asn (leader A). These junctions (exon ⅓ and exon ⅔) are positioned 2 aa from the N terminus of the mature Ch 1, which starts with Glu¹ . The structural gene is comprised of only two exons, 3 and 4, that encode the mature protein (FIG. 2).

Ch 2 is composed of a mature protein subunit of 92 aa. The leader sequence and the first 3 aa of the mature protein are encoded by exon 1 (61 nt; 20 aa). The bulk of the mature protein is encoded by exons 2 and 3 (aa 4-64 and 65-90, respectively). The first 18 nt of exon 3 encode the residues, IAINEY (aa 65-70), rather than the published sequence, TTISSSKD, suggesting that Ch2 has two forms (FIG. 3).

While any of the exons can be targeted by the vector construct, it is preferential to allow for at least 1000 bp of homology on either side of the targeted exon. It has been demonstrated that this contributes to a greater success rate of recombination events.

C. Vector Design

1. General Vector Design With Selectable Mutations

Generally, gene targeting by homologous recombination occurs at a low frequency in comparison to random integration events. For most genes, vectors can be designed to reduce the frequency of random integration events surviving selection. A gene that is expressed in ES cells can be targeted using a selectable marker with no promoter. The selectable marker can either have its own translation initiation signal or form a fusion protein with the targeted gene. Alternatively, the selectable marker can be placed within the gene so that the polyadenylation signal must be supplied by the genomic integration site.

For any gene, a negative selectable marker (i.e., strp^{s}) can be used outside the homologous region in the targeting vector. In a correct targeting event, the negative selectable marker will be excised and the cells will be resistant to streptomycin, but in the random events, the negative marker will generally be integrated and expressed, causing cell death via metabolism of the toxic nucleoside analog. These strategies can be used alone or in combination to help increase the relative gene targeting frequency. The number of clones with random integration events that survive selection will be reduced which will make the targeted event easier to detect.

The factors that determine the frequency with which a genomic locus will be targeted have not as yet been determined completely. Factors which do affect the targeting frequency include the length of perfect homology between the targeting vector and the genomic locus, the placement of the selectable marker within the homologous stretch, and the site of linearization of the vector. The standard replacement vector using positive-negative selection has shown targeting frequencies of 1/10 to 1/1000 G418^{r}- strp^{s} colonies for many genes. Regarding the length of homologous sequences in the targeting vector, a convenient compromise between vector construction, diagnosis of targeted events, and targeting frequency is 3 kb with at least 1 kb on either side of the selectable marker. It is best to construct the targeting vector with DNA from the same cat strain as the ES cell line since polymorphisms could disrupt the length of perfect homology and result in a lower targeting frequency. Careful consideration should be given to the structure of the locus after the desired recombination event, especially if a null allele is desired. For small genes, replacement vectors can be designed in which the coding sequence is replaced by the selectable marker. For larger genes, disruption of the first coding exon is most likely to give a null allele.

A Fel d I gene can also be disrupted, and inactivation, by deletion of all or part of the Fel d I gene, so as to prevent production of a functional Fel d I protein.

500 colonies are routinely screened by “mini-Southern” analysis (Section F) after the first round of targeting. If targeted clones are found, they should be examined by several digests on Southern analysis using probes and enzymes specific for both the 5′ and the 3′ ends of the homologous sequences, to ensure that the desired recombination event has occurred. If clones are not identified, it is best to redesign the vector rather than continue further screening.

Insertion vectors have been shown to target between 5- and 12-fold more frequently than replacement vectors and could be used for subsequent attempts at targeting. Depending on the design of the original replacement vector, it may be possible to linearize the same vector within the area of homology to take advantage of the higher targeting frequency of insertion events. For a general discussion of vector design, see Ramirez-Solis et al., Methods in Enzymology, 1993.

2. Fel d I Vector Design

Fel d I has the advantage of having two genes that code for the major allergen. This means that constructs can be designed to disrupt the coding sequence of either chain 1 (Ch 1), chain (Ch 2), or both chains. For a general discussion of site directed mutagenesis of target genes, see Thomas and Capecchi, “Site-Directed Mutagenesis by Gene Targeting in Mouse Embryo-Derived Stem Cells” Cell (1987).

A specialized construct of the neomycin resistance (neo^{r}) gene is introduced into one of the exons of a cloned fragment of either Ch 1 or Ch2. This construct is then used to transfect the ES Cells. The neo^{r} gene is used both to disrupt the coding sequence of the target gene and as a tag to monitor the integration of the newly introduced DNA into the recipient genome. Effective use of the neo^{r} gene as a tag requires expression of the gene at the appropriate Fel d I locus.

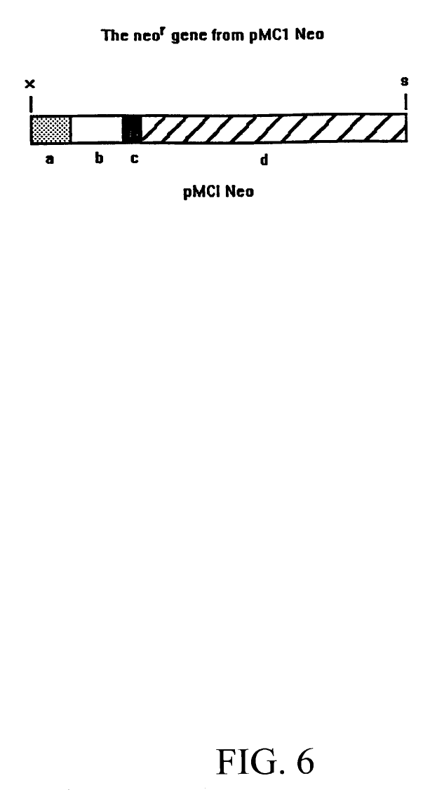

The neomycin gene is designed to optimize expression in ES cells while maintaining its size at a minimum. The neo^{r} has been modified for this purpose and is designated pMClNeo, and the overall structure for this construct is shown in FIG. 6. The neomycin protein coding sequence (d) is from the bacterial transposon Tn5, including bases 1555-2347. The promoter (b) that drives the neo^{r} gene is derived from the herpes simplex virus thymidine kinase gene (HSV-tk) from bases 92-218. This promoter appears to be effective in embryonal carcinoma (EC) cells. To increase the efficiency of the tk promoter, a duplication of a synthetic 65 bp fragment (a) consisting of bases 5210-5247 of the PyF441 polyoma virus enhancer is introduced. This fragment encompasses the DNA sequence change that allows the polyoma mutant to productively infect EC cells. Finally, because the native neo^{r} gene translation initiation signal is particularly unfavorable for mammalian translation, a synthetic translation initiation sequence (c) (GCCAATATGGGATCGGCC) is substituted using Kozak's rules as a guide (Kozak, 1986) (FIG. 6). See Thomas and Capecchi, supra for a discussion of this construct.

There are two schemes to disrupt the Fel d I genes: one by sequence replacement vectors and one by sequence insertion vectors. Both vectors contain an exon interrupted with the neo^{r} gene.

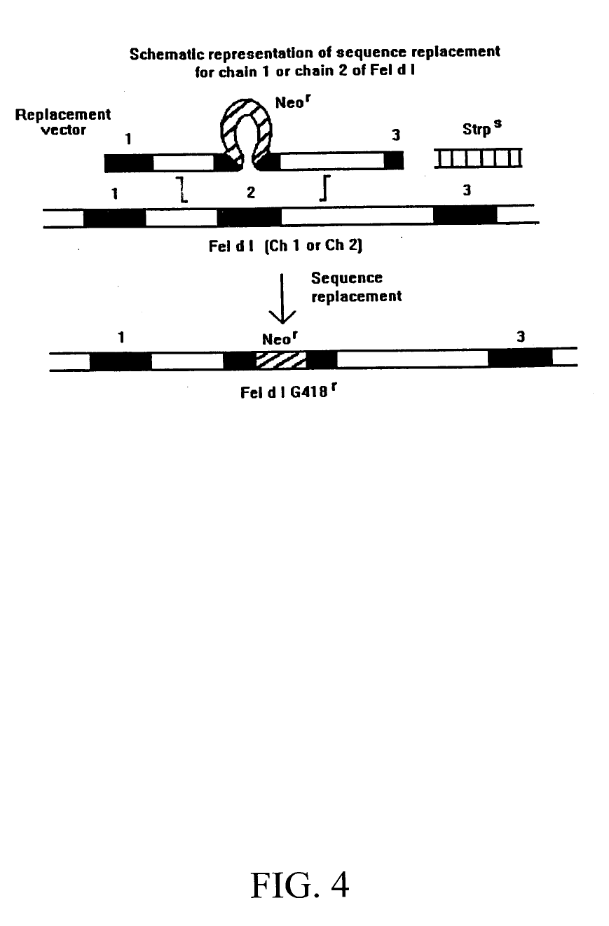

Sequence replacement vectors are designed such that upon linearization, the vector sequences remain collinear with the endogenous sequences. Following homologous pairing between vector and genomic sequences, a recombination event replaces the genomic sequences with the vector sequences containing the neo^{r} gene (FIG. 4).

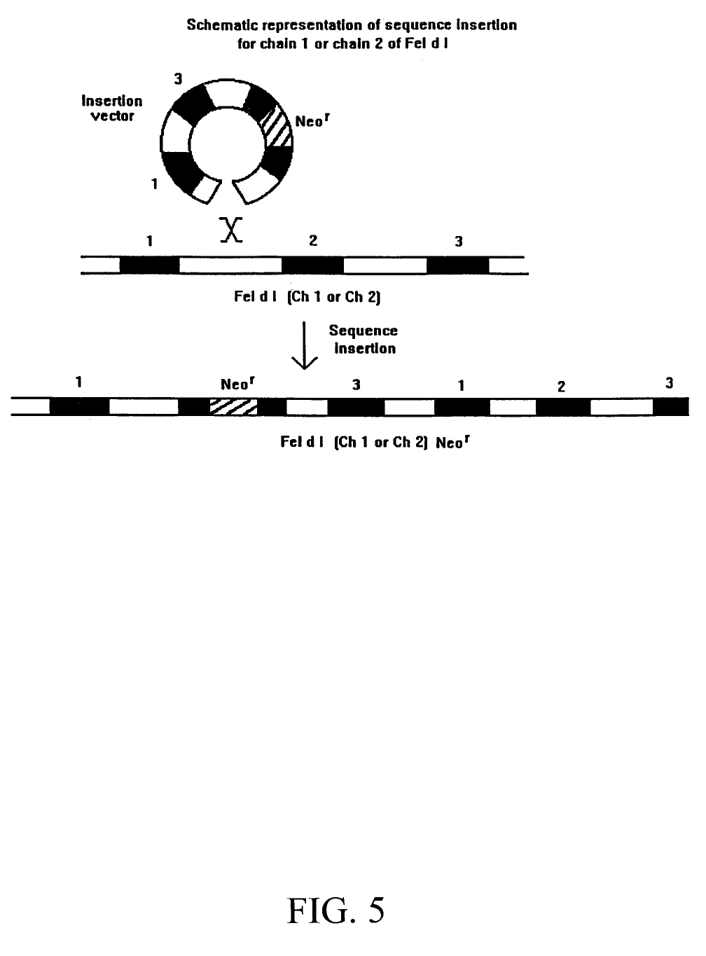

Sequence insertion vectors are designed such that the ends of the linearized vector lie adjacent to one another on the gene map. Pairing of these vectors with their genomic homolog, followed by recombination at the double strand break, results in the entire vector being inserted into the endogenous gene (FIG. 5).

Successful homologous recombination after electroporation renders the ES cells resistant to the drug G418r. To make initial screening easier, a streptomycin sensitive gene can be added outside of the homologous coding region of the replacement vector. Upon successful gene replacement, this stfp^{s} gene is lost and ES cell colonies will grow on media containing streptomycin. If the recombination is random in the genomic DNA, the strp^{s} gene will be retained and the ES cells will not grow.

D. Electroporation

The first step of any targeting experiment is the introduction of DNA into the recipient cells. For ES cells, DNA microinjection and electroporation have been shown to be useful to permit gene targeting. DNA microinjection is technically difficult and has the potential to cause gross chromosomal disruption, which may lower the potential of the ES cells to populate the germ line of chimeras. Electroporation, on the other hand, has been used extensively to generate targeted clones that have gone through the germ line. The electroporation protocol used is basically similar to those used for other cell types, but some things are particularly important for the specific case of electroporation of ES cells. The cells should be growing actively at the time of the electroporation; this can be achieved by passaging the ES cells 1 day before the electroporation and adding fresh medium a few hours before harvesting the cells. The trypsin treatment should be long enough to allow mechanical disaggregation of the cell clumps to avoid differentiation. The electroporated cells should be plated on feeder cells with M15 medium within 5-10 min. The following procedure, described in Ramirez-Solis et al., Methods in Enzymology, 1993, can be used:

1. Prepare targeting vector DNA by the CsCl banding technique.

2. Cut 200 ug of targeting vector DNA with the appropriate restriction enzyme to linearize it. Assess the completion of the restriction digest by agarose gel electrophoresis.

3. Clean the DNA with phenol-chloroform, chloroform, and precipitate it with NaCl and ethanol. Resuspend the DNA in sterile 0.1×Tris-EDTA buffer (TE) and adjust the concentration to 1 mg/ml.

4. One day before the electroporation, passage the actively growing ES cells (˜80% confluent) 1:2.

5. Feed the cells with fresh M15 medium 4 hr before harvesting them for the electroporation.

6. Wash the plates twice with PBS and detach the cells by treatment with trypsin solution for 10 min at 37° (1 ml trypsin solution for a 10-cm plate).

7. Stop the action of the trypsin solution by adding 1 volume of M15 medium and dissociate the cell clumps by moving the cell suspension up and down with the transfer pipette.

8. Centrifuge the cells at 1000 rpm for 5 min in a clinical centrifuge and discard the supernatant. Resuspend the cells in 10 ml of PBS and determine the total number of cells.

9. Recentrifuge the cells, aspirate the supernatant, and resuspend the cells in PBS at a final density of 1.1×10⁷ cells/ml.

10. Mix 25 ug of the linearized targeting vector with 0.9 ml of the cell suspension in an electroporation cuvette. Incubate for 5 min at room temperature.

11. Electroporate in the Bio-Rad Gene Pulser at 230 V, 500 uF. Incubate for 5 min at room temperature.

12. Plate the entire contents of the cuvette on a 10-cm tissue culture plate with feeder cells. The medium on the feeder plate should be changed to M15 prior to plating the cells.

13. Apply G418 selection 24 hr after the electroporation. FIAU selection can also be applied if a positive-negative selection protocol using the herpes simplex virus- 1 thymidine kinase (HSV-1 tk) gene is being followed.

14. Refeed the cells when the medium starts turning yellow, usually daily for the first 5 days.

15. Ten days after the electroporation, the colonies are ready to be picked.

E. Picking and Expansion of Colonies after Electroporation

After electroporation, the ES cell colonies take 8-12 days of growth to become visible to the naked eye and can be picked at this time. Care should be taken that only a single colony is seeded per well to avoid a further cloning step. See Ramirez-Soliset al., Methods in Enzymology, 1993.

1. Wash the plate containing the colonies twice with PBS and add PBS to cover the plate.

2. Prepare a 96-well U-bottomed plate by adding 25 ul of trypsin solution per well.

3. Place the original 10-cm plate on an inverted microscope and pick individual colonies with a micropipettor and disposable sterile tips in a maximum volume of 10 ul. Each colony is transferred to the trypsin solution in a well of the plate prepared in Step 2.

4. After 96 colonies have been picked, place the 96-well plate in the 37°, 5% CO₂ incubator for 10 min.

5. During the incubation, take a previously prepared 96-well feeder plate (flat-bottomed wells), aspirate the medium, and add 150ul of M15 per well. Use a multichannel pipettor (12 channels) for all following steps.

6. Retrieve the trypsinized colonies from the incubator and add 25 ul of M15 per well. Break up the clumps of cells by moving the cell suspension up and down with the multichannel pipettor about 5-10 times.

7. Transfer the entire contents of each well to a well in a 96-well plate prepared in Step 5. Change tips each time.

8. Put the plate in the incubator and grow for 3-5 days, changing the medium as necessary.

9. When the wells are approaching confluence, wash twice with PBS and trypsinize using 50 ul of trypsin solution per well during 10 min. Add 50 ul of M15 and break up cell clumps by vigorous pipetting. Replate 50 ul onto a gelatinized 96-well plate without feeder cells. The remaining cells in the original 96-well plate may be frozen by adding 50 ul of 2× freezing medium and proceeding through the next protocol from Step 4.

The gelatinized plate can be grown to confluence for DNA preparation and analysis by “mini-Southem” blotting (Section G). Once the targeted clones have been identified, the appropriate wells can be retrieved from the freezer and expanded for blastocyst injection and further DNA analysis (Section F).

F. Freezing and Thawing ES Cells in 96-Well Plates

Freezing ES clones in individual vials while screening for targeted clones is laborious and time-consuming work, especially if the number of clones to be screened is very large. A strategy has been devised to freeze ES cells in 96-well tissue culture dishes that consistently allows a recovery of 100% of the thawed clones. See Ramirez-Solis et al., Methods in Enzymology, 1993.

1. Change the medium on the cells 4 hr before freezing.

2. Discard the M15 medium by aspiration and rinse the cells twice with PBS.

3. Add 50 ul of trypsin solution per well with the multichannel pipettor and incubate the plate for 10 min at 37°, 5% CO₂.

4. Add 50 ul of 2× freezing medium per well and dissociate the colonies.

5. Add 100 ul of sterile light paraffin oil per well to prevent degassing and evaporation during storage at −70°.

6. Seal the 96-well plate with Parafilm and put it into a Styrofoam box; close the box and store it at −70° for at least 24 hr. For long-term storage, transfer the plate to a minus 135° freezer.

7. To thaw, take the 96-well plate out of the freezer and place it into the 37° incubator for 10-15 min.

8. Identify the selected clones and put the entire contents of the well into a 1-cm plate (24-well) with feeder cells containing 2 ml of M 15 medium. Change the medium the next day to remove the DMSO and the oil.

G. Southern Blot Analysis Using DNA Prepared Directly on Multiwell Plates

Screening by Southern blotting necessitates that the colonies be expanded in vitro to provide enough DNA to carry out such an analysis. In this context, it is very important to increase the efficiency of DNA recovery during the extraction process, which will consequently diminish the time that the cells have to be expanded. A replica of the clones may be frozen while carrying out the analysis. A protocol to freeze cells directly in a 96-well plate has been given (Section F). To further improve the efficiency of the gene targeting protocol, a DNA extraction technique that provides a fast, simple, and reliable way to screen a large number of clones by Southern analysis has been developed. After the cell suspensions have been divided into halves and one-half has been frozen, the other is plated on a gelatin-coated 96-well replica plate (Section E). This last plate provides the initial material for the DNA microextraction procedure. Lysis of the cells is carried out in the plate by adding lysis buffer and incubating overnight at 60° in a humid atmosphere. The nucleic acids are precipitated in the plate and remain attached to it while the solution is discarded by simply inverting the plate; the nucleic acids are then rinsed, dried, and the DNA cut with restriction enzymes in the plate. All 96 samples can be separated by electrophoresis in a single gel. This greatly accelerates the rate at which screening can be done by Southern blotting. This protocol has been tested for several restriction enzymes, and all give complete DNA restriction using this procedure. However, a pilot reaction with the enzyme of choice should be performed before starting a large screen. When handling a large number of plates, label bottoms and lids to avoid confusion. See Ramirez-Solis et al., Methods in Enzymology, 1993.

1. Allow the cells on the gelatin-coated plates to grow until they turn the medium yellow every day (4-5 days).

2. When the cells are ready for the DNA extraction procedure, rinse the wells twice with PBS and add 50 ul of lysis buffer per well.

3. Incubate the plates overnight at 60° in a humid atmosphere. This is easily achieved by incubating the plates inside a closed container (Tupperware) with wet paper towels in a conventional 60° oven.

4. The next day, add 100 ul per well of a mix of NaCl and ethanol (150 ul of 5M NaCl to 10 ml of cold absolute ethanol) using a multichannel pipettor.

5. Allow the 96-well plate to stand on the bench for 30 min at room temperature without mixing. The nucleic acids precipitate as a filamentous network.

6. Invert the plate carefully to discard the solution; the nucleic acids remain attached to the plate. Blot the excess liquid on paper towels.

7. Rinse the nucleic acid 3 times by dripping 150 ul of 70% ethanol per well using the multichannel pipettor. Discard the alcohol by inversion of the plate each time.

8. After the final wash, invert the plate and allow it to dry on the bench. The DNA is ready to be cut with restriction enzymes.

9. Prepare a restriction digestion mix containing the following: 1× restriction buffer, 1 mM spermidine, bovine serum albumin (BSA, 100 ug/ml), RNase (100 ug/ml), and 10 units of each restriction enzyme per sample.

10. Add 30 ul of restriction digest mix per well with a multichannel pipettor; mix the contents of the well using the pipette tip and incubate the reaction at 37° overnight in a humid atmosphere.

11. Add gel electrophoresis loading buffer to the samples and proceed to conventional electrophoresis and DNA transfer to blotting membranes. Use a 6 by 10 inch 1% (w/v) agarose gel with three 33-tooth combs spaced 3.3 inches apart. This gives enough space for 96 samples plus one molecular weight marker lane for every comb. Gel electrophoresis in 1×TAE at 80 V for 4-5 hr gives a good separation in the 1-10 kb range.

H. Freezing and Thawing Embryonic Stem Cells in Vials

Clones that appear to have the desired mutation should be expanded and frozen in vials. See Ramirez-Solis et al., Methods in Enzymology, 1993.

1. Dissociate the cells that have been expanded in the 1-cm plate (Section E) with 0.2 ml of trypsin solution for 10 min at 37°, then stop the action of the trypsin by adding 1 volume of M 15 and disaggregate the cell clumps as mentioned before.

2. Take the necessary cells for blastocyst injection and for expansion for further DNA analysis, and freeze the rest as follows.

3. Slowly add 1 volume of 2× freezing medium and mix the cell suspension gently.

4. Distribute the cell suspension into aliquots in sterile freezing vials. Place the vials in a Styrofoam container, close it, and store it at −70° overnight. The next day, transfer the vials to a −135° freezer, or to liquid nitrogen.

5. To thaw, transfer the vial containing the frozen cells to a 37° water bath.

6. When the cell suspension has thawed, transfer it to a sterile 15-ml tube. Add M15 medium slowly, while shaking the tube; fill the tube with M15 medium and collect the cells by centrifugation at 1000 rpm for 5 min at room temperature.

7. Discard the supernatant by aspiration, resuspend the cell pellet in 2 ml of M15 medium, ensure the absence of cell clumps, and plate the cell suspension onto a 1-cm plate with feeder cells. Incubate at 37°.

IV. Getting Mutations into the Germ Line

The protocols described to date have all had the aim of generating a mutation in ES cells in such a way that the cells remain totipotent and can thus contribute both to somatic tissues and, most importantly, to the germ line of a cat. Thus, it is important always to grow ES cells on feeder layers, to keep the time in culture to a minimum (particularly at low density), and to dissociate clumps of cells at each passage. To test the pluripotency of each targeted clone, sufficient blastocysts should be injected to give two litters. The sex of the offspring should be determined.

The ES cell lines are usually derived from male blastocysts, and extensive contribution to the injected embryo will convert a female blastocyst to a male animal. This gives a disproportionate number of males in the litter. In addition, males that are converted female blastocysts are desirable, as they transmit only ES cell-derived genes to their offspring. They often have reduced fertility, but this disadvantage is more than offset by the efficient transmission of the mutation by the fertile animal. Experience indicates that if a clone does not give high ES cell contribution chimeras or a good sex distortion in 10-12 offspring, then repeated injections of that clone are unlikely to result in germ line transmission. Male chimeras from those clones should be test bred. Ideally, for any mutation, two clones should be established in the germ line to confirm that the phenotype is the result of the engineered change. Under ideal conditions, 80-90% of injected clones should be transmitted through the germ line. For general discussion of techniques, see Ramirez-Solis et al., Methods in Enzymology, 1993.

A. Aggregation of 8-Cell Stage Embryos with Embryonic Stem Cells

The following procedure is adapted from a protocol described in Stewart, “Production of chimeras Between Embryonic Stem Cells and Embryos.” Methods in Enzymology, 1993.

Presently, there are three methods of producing ES cell chimeras: (1) blastocyst injection, (2) morula injection, and (3) morula aggregation. This protocol will use morula aggregation.

All that is necessary for the aggregation procedure is a good stereo dissection microscope with magnification to 40 ×and a mouth-controlled micropipette. This procedure has also been modified to produce embryos/cats that are entirely derived from the ES cells. This involves the aggregation of ES cells with two tetraploid 4-cell stage embryos. Tetraploid embryos are routinely produced by electrofusion of diploid blastomeres at the 2-cell. Aggregating the diploid ES cells with tetraploid blastomeres results in the ES cells forming most of the ICM, whereas derivatives of the tetraploid embryos tend to form the extraembryonic membranes such as the trophectoderm and yolk sac endoderm. Thus, at birth, the embryo derived from the ICM will be largely or entirely derived from the ES cells. The extraembryonic membranes derived from the tetraploid embryos, in the form of the placenta and yolk sac, are lost at birth.

B. Preparation of 8-Cell Stage Embryos for Aggregation

1. The surgical recovery of embryos are performed by uterine lavage between day 11 and day 13 after onset of FSH and hCG treatment. 8-cell stage embryos are isolated. The embryos are washed twice in M2 to remove any cellular debris, blood cells, etc., and are cultured in drops of CZB plus glucose medium under paraffin oil. See Stewart, supra.

The following steps are described in Verstegen, Journals of Reproduction and Fertility, 1993:

2. To aggregate ES cells with the embryos, it is necessary to remove the zona pellucida. This is done by incubating the embryos for 20-40 sec in dishes of prewarmed (37^{O}) acidified Tyrode's solution. In batches of 10, the 8-cell stage embryos should be introduced into a 35-mm dish containing acidified Tyrode's solution. The low pH of the Tyrode's solution results in the zona pellucida dissolving in the saline solution. The acidified Tyrode's solution should be between pH 2 and 3, if the embryos are to be completely freed of their zonae. As soon as the zona has disappeared, the embryos are removed from the Tyrode's solution and washed 3 times in M2 medium.

3. In a 60-mm bacteriological grade petri dish, set up three 20-ul drops of medium containing a 50:50 mixture of DMEM plus 10% FCS and CZB plus glucose. In addition, set up 20 1-ul drops of the same medium. Cover with light paraffin oil. The three 20-ul drops will hold the ES clumps (see below) that will be aggregated with the embryos. Into each 1-ul drop of medium, transfer two 8-cell stage embryos. The benefit of the small drops is that they not only provide sufficient nutrients for overnight culture, but also physically confine the embryos. When 20 pairs have been set up, the dish is returned to the incubator.

C. Preparation of Embryonic Stem Cells for Aggregation

The following procedure is described in Stewart, supra.

1. The ES cells are prepared as small aggregates of between 5 and 10 cells each rather than single cells (which would be difficult to manipulate).

2. A 35- or 60-mm dish of ES cells, in which the cells are growing (in the log phase) as colonies on feeders, is washed twice in Ca²⁺/Mg²⁺-free PBS. The cells are then covered in Ca²⁺/Mg²⁺-free PBS containing 0.5 mM EGTA and left for 5 min. This causes the cells in the colonies to loosen their attachment to each other. The loosened colonies of ES cells are drawn up using a mouth-controlled pipette having an internal opening diameter of about 50-75 um with the edges of the tip smoothed by flame polishing. The colonies are then transferred to 20-ul microdrops of 50:50 DMEM plus 10% FCS and CZB medium. By gently blowing the colonies back and forth between the pipette and microdrops, the colonies will fall apart into clumps of ES cells. The clumps are allowed to settle onto the surface of the dish. Individual clumps of 5-10 cells are selected and then introduced into the 1-ul drops containing the two 8-cell stage embryos.

3. The aggregation procedure consists of using a mouth-controlled pipette to push the clump of ES cells into a crevice between two blastomeres. It is important to ensure that the embryos have not started to compact because aggregation with uncompacted embryos is easier and usually results in the clump of cells adhering to the blastomeres. The second embryo is then maneuvered by the pushing/gentle blowing of medium into a position so that it sandwiches the ES clump that is attached to the first embryo. Both embryos must be in contact with each other. Adherence and subsequent aggregation of the ES cells to the embryos are temperature-dependent, and the whole process is more difficult if the dish and embryos are allowed to cool substantially. When all the embryos have been aggregated, the dish is returned to the incubator. Fifteen to twenty minutes later, each aggregate should be checked to ensure that the embryos are still attached to each other and to a clump of ES cells. If a clump of ES cells is not adhering to the embryo (this can be determined by gently blowing the whole aggregate around the microdrop to ensure that all components are sticking to each other), replace the cells with another group. The aggregated ES cells/embryos are then cultured overnight. The following morning, the majority of aggregates should have formed blastocysts. These are then surgically transferred to the uteri of pseudopregnant recipients.

V. Transfer of Embryos to Pseudopregnant Recipient

A. Preparation of Pseudopregnant Recipients

For manipulated embryos to develop to term, they have to be returned to the uterus for proper implantation and development. Female cats must be mated with males for them to initiate the physiological changes associated with pregnancy. If females are mated to normal males, they would contain viable embryos resulting from that mating. The presence of these embryos would compete with any experimentally manipulated embryos transferred to the uteri of the pregnant female. To avoid this but to still induce pregnancy, female recipients are mated with vasectomized males, which can mate with females but cannot fertilize eggs. See Stewart, supra.

B. Vasectomizing Male Cats

The following procedure is described in Stewart, supra.

1. Anesthetize a 4 to 6 month old male cat (Taylor, The Ultimate Cat Book, Dorling and Kindersley Ltd., N.Y., N.Y., 1989) by a single injection of Avertin. To make Avertin add 0.5 g of 2,2,2-tribromoethanol to 0.63 ml of tert-amyl alcohol prepared in a 1-ml Eppendorf tube. Vortex to dissolve the tribromoethanol. Add 0.5 ml of this solution to 19.5 ml of prewarmed 0.9% saline solution, in which the anesthetic will dissolve after shaking, and allow to cool. The dose injected is 0.012 ml/g body weight.

2. The anesthetized male is laid on its back, the belly is swabbed with 70% ethanol solution, and a horizontal incision using scissors is made through the skin. All surgical procedures should be performed under a stereo dissection microscope with an incident light source.

3. Expose the underlying peritoneum and make a horizontal incision. This should expose two fat pads.

4. Using a pair of blunt forceps, grasp one of the fat pads and pull it out of the body cavity. This results in the testis also being pulled out with it. Beneath the fat pad and connected to the testis is a muscular tube, the vas deferens. This can be recognized by the single blood vessel that runs along its side.

5. Using a pair of fine forceps, a loop is made in the vas deferens. With a pair of forceps, the tips having been preheated, the loop of vas deferens is cauterized and severed. This results in a section of the tissue being removed, with the remaining ends being sealed.

6. The testis/fat pad is then gently moved back into the peritoneal cavity, and the process is repeated for the other testis.

7. Once the procedure is completed, the peritoneal incision is ligated together using a surgical needle and thread. The skin cut is then clamped together using wound clips.

8. The male is allowed to recover. The animal should be set up and test-mated with females to ensure sterility. The wound clips should be removed 10-14 days after the operation.

C. Transfer of Manipulated Embryos to Pseudopregnant Recipients

For the injected/aggregated embryos to develop to term, they have to be transferred to the uteri of pseudopregnant recipients (i.e., females mated with vasectomized males). For Morula injection/aggregation, transfer occurs the following day, that is, once they have developed to the blastocyst stage, which follows overnight culture in vitro. See Stewart, supra.

It is best to transfer the blastocysts to pseudopregnant recipients whose stage of pregnancy is 1 day behind that of the blastocyst. In normal pregnancy, blastocysts are found in the uteri of day 13 pregnant cats, so the manipulated embryos are transferred to the uteri of day 12 pseudopregnant recipients. This apparently gives blastocysts time to recover in vivo from the in vitro manipulations (Verstegen, Journals of Reproduction and Fertility, 1993). Transfer to day 12 recipients also results in a higher incidence of implantation than when blastocysts are transferred to synchronized recipients (i.e., day 12 pregnant females).

If possible, 6-7 embryos should be transferred to each uterine horn. If fewer are available, then transferring to only 1 horn is satisfactory.

1. Female cats that were mated 12 days previously with vasectomized males are anesthetized by an injection of Avertin. Females should be between 18 and 36 months in age (Taylor, The Ultimate Cat Book, Dorling and Kindersley Ltd., N.Y., N.Y, 1989).

2. After weighing, the female is injected intraperitoneally with the appropriate volume of Avertine (see section on vasectomizing male cats). The animal should be fully anesthetized within 2-3 min, which is deter-mined by gently squeezing one of the rear paws. If the animal responds by rapidly shaking back and forth, the animal is not anesthetized and needs to be left longer for the anesthetic to take its full effect or be given an additional injection of about one-third the original dose.

3. Once fully anesthetized, the female is laid on its back, the belly is swabbed with 70% ethanol solution, and a horizontal incision using scissors is made through the skin. All surgical procedures should be performed under a stereo dissection microscope with an incident light source. The incision is opened, and some of the transparent mesentery attaching the skin to the peritoneum lying immediately beneath the skin is cut or pulled away. The skin incision is moved over the peritoneum to the point where the right ovary is seen to be lying just beneath the peritoneum. The ovary is recognized by its bright cherry red color (owing to the numerous copora lutea). An incision of no more than 0.5 cm is made through the peritoneum, with care being taken to avoid cutting any of the blood vessels visible in the peritoneum. The ovary is attached to a fat pad and to the oviduct and uterus. By grasping the fat pad, the ovary, oviduct, and uterus are pulled out of the peritoneal cavity with a pair of blunt forceps, exposing the ovarian end of the uterus. To keep the uterus from sliding back into the peritoneal cavity, the fat pad is clamped with a small pair of aneurism clips, which is of sufficient weight to prevent the organ from sliding back. It is important that the uterus not be touched during the surgical procedure, since trauma may result in failure of the embryos to implant.

4. With the ovarian end of the uterus lying on the peritoneum wall, a hole is made in the uterus just above the uterine-oviduct junction, using a new (sterile) 25-gauge syringe needle. It is only necessary to penetrate the wall of the uterus using the tip of this extremely sharp needle, which should be inserted no more than 1-2 mm.

5. The blastocysts to be transferred have, at this point, already been picked up and are lying in the transfer pipette. These pipettes can be readily pulled on a gas or alcohol burner flame. The internal diameter should be about 100um, and the tip should be no longer than 2-4 cm. Light paraffin oil is drawn into the barrel of the pipette using mouth. The viscosity of the paraffin oil gives a much finer level of control in pipetting medium, which is required for picking up and transferring the blastocysts into the uterine lumen. The embryos to be used for transfer are sitting in a 35-mm dish of prewarmed M2 medium with no paraffin oil covering the medium. The transfer pipette, with the tip filled with paraffin oil, is introduced into the M2 medium. A small amount of medium is drawn up into the tip, followed by a small air bubble. More medium is taken up at about 0.5-1 cm, and then a second small air bubble. This is followed by drawing up 6-7 blastocysts in as small a volume of M2 medium as possible, followed by a third air bubble. The air bubbles act as markers for determining where the embryos are lying, since they are more visible in the pipette than the embryos. The two lowermost bubbles, which sandwich the embryos, indicate where the embryos are lying in the pipette. The first, uppermost bubble acts as a marker to indicate when all the embryos have been transferred into the uterus.

6. Using a pair of fine forceps, grasp the oviduct to steady the uterus. The tip of the transfer pipette is inserted into the hole in the uterine wall and is pushed about 3-5 mm into the uterine lumen. This should be done gently; any resistance indicates that the tip is in contact with the uterine endometrium. Once the transfer pipette has been inserted sufficiently deep into the uterus, it is withdrawn about 1-2 mm to ensure that the opening at the tip (still within the lumen) is not in contact with the endometrium, which would block the exit of embryos into the uterine lumen. The embryos are expelled into the lumen, with the transfer being followed by watching the air bubbles. When the last air bubble (i.e., the one nearest the paraffin oil) is seen to enter the uterus, the pipette is withdrawn. The tip is immediately placed into the dish containing the remaining blastocysts, and medium is gently drawn back and forth through the tip. This cleans any blood that may be adhering to the tip which, if clotted, will block the tip. This washing also ensures that all the embryos were transferred to the uterus. The next set of blastocysts can then be picked up in the transfer pipette using the same arrangement of medium and air bubbles.

7. The uterus into which the embryos were transferred is gently pushed back into the peritoneal cavity after the ancurism clip is removed from the fat pad. The wall is pinched together and can be sutured, although this is not usually necessary. The process is repeated for the remaining uterine horn. When the operation is completed, the edges of the skin where the incisions were made are stapled together by two or three 0.9-mm wound clips (Clay Adams, Becton-Dickinson and Co., Parsippany, N.J.). The recipients are placed on a 37° warmer to keep the cats warm until they regain consciousness. The manipulated embryos should be born within 60-70 days of the day of transfer (Taylor, The Ultimate Cat Book, Dorling and Kindersley Ltd., N.Y, N.Y, 1989).

It is possible to knock our both alleles at the ES cell level and generate the homozygous animal directly. Normally, however, the heterozygote cell is injected, and the cats carrying the desired targeted locus are then bred to produce a homozygote See generally, Robbins, Circulation Research 73:3-9 (1993).

Having described the preferred embodiments of the present invention, it will appear to those ordinarily skilled in the art that various modifications may be made to the disclosed embodiments, and that such modifications are intended to be within the scope of the present invention.

VI. Generation of Allergen-free Transgenic Animals Using Other Techniques

While the above procedure describes the use of embryonic stem cells in the production of allergen-free animals, there are other cloning techniques that can be used to create transgenic animals. One such technique that has enjoyed recent success is nuclear transfer. For example, Sims et al., (1993), Proc. Natl. Acad. Sci. USA 90:6143-6147 produced calves by transfer of nuclei from cultured inner cell mass cells; Wilmut et al. (1997), Nature 385:810 and Schnieke et al. (1997) Science 278:2130 demonstrated that nuclei from fetal fibroblast cells have directed the formation of lambs; Cibelli et al. (1998) Science 280:1256 cloned cattle cow calves using nuclei from fetal fibroblast cells; Wakayama et al. (1998) Nature 394:369 used nuclear transfer to produce fertile mice from cumulus cells collected from metaphase II oocytes; and most recently Kato et al., (1998), Science 282:2095-2098 using nuclear transfer technology cloned eight calves from cumulus cells and oviductal cells of a single adult.

In this procedure, the DNA from mature somatic cells can be altered, for example, by transfecting the mature somatic cells with a targeting vector comprising an inactivated allergen gene. When the gene inactivation is confirmed, the donor cells are rendered quiescent in the G₀-G₁ phase by serum starvation for 3-4 days. These techniques are well-known in the art, see, for example, Wilmut et al. (1997), Nature 385:810 and Kato et al., (1998), Science 282:2095-2098, which are specifically incorporated herein by reference. Then these donor cells are fused with enucleated oocytes from the same animal species. Molecules within the embryonic environment cause the differentiated mature DNA to revert back to embryonic DNA. These cells then begin to divide as though they were a part of a newly developing embryo. Thus the derived nuclear transplants are cultured in vitro into blastocysts which are transferred, surgically as described above, or non-surgically, into surrogate mothers at an appropriate time after the onset of estrous. The resulted pregnancy are allowed to carry to term and transgenic animals are delivered, preferably vaginally or with surgical assistance, using established techniques well-know in the art.

Thus, in accordance to one embodiment of the invention, a transgenic non-human vertegrate animal are produced, wherein the genome of said animal comprises an allergen gene that is inactivated. More preferably, the transgenic animal according to the invention does not produce functional product of said allergen gene. According to another embodiment of the invention, the allergen gene of both the somatic cells and the germ line cells the transgenic animal so produced are inactivated.

According to another embodiment of the invention, the transgenic animal is fertile and capable of transmitting said inactivated allergen gene to its offspring.

The invention also teaches a method for producing a transgenic non-human vertebrate animal comprising an inactivated allergen gene, said method comprising: (a) introducing an animal stem cell comprising an inactivated allergen gene into an animal embryo; (b) transplanting said animal embryo into a pseudopregnant animal; and (c) allowing said animal embryo to mature into an animal.

According to the invention, another preferred method for producing a transgenic non-human vertebrate that comprises an inactivated allergen gene, that does not produce said allergen, and that is homozygous for said inactivated allergen gene, comprises (a) introducing an inactivated animal allergen gene into a cell of said animal; (b) selecting for an animal cell that comprises only the inactivated allergen gene, but not a functional allergen gene; (c) isolating the nucleus of said cell of step (b) comprising the inactivated allergen gene; (d) transferring the nucleus of step (c) into an enucleate egg cell of said animal; (e) transplanting said egg into a pseudopregnant animal and render the animal pregnant; and (f) carrying the pregnancy to term and obtain a transgenic animal.

What is claimed is:

1. An isolated polynucleotide sequence encoding a disrupted Fel d I gene.

2. A sequence according to claim 1, wherein said Fel d I gene has been disrupted by sequence replacement.

3. A sequence according to claim 1, wherein said Fel d I gene has been disrupted by sequence insertion.

4. A sequence according to claim 1, wherein said Fel d I gene has been disrupted by deletion of all or a part of said Fel d I gene.

5. A sequence according to claim 1, wherein said Fel d I gene has been interrupted with a polynucleotide sequence encoding a selectable marker.

6. A sequence according to claim 5, wherein said selectable marker is a gene that confers neomycin resistance.

7. A recombinant polynucleotide vector comprising all or part of a disrupted Fel d I gene.

8. An embryonic cat stem cell comprising a sequence according to claim 1.

9. An embryonic cat stem cell comprising a vector according to claim 7.

10. A transgenic cat comprising a disrupted Fel d I gene.

11. A cat according to claim 10, wherein the Fel d I gene of the somatic cells of said cat is disrupted.

12. A cat according to claim 10, wherein the Fel d I gene of the germ line cells of said cat is disrupted.

13. A cat according to claim 10, wherein the Fel d I gene of the germ line cells and the somatic cells of said cat is disrupted.

14. A cat according to claim 10, wherein said cat is heterozygous for the disrupted Fel d I allergen gene.

15. A cat according to claim 10, wherein said cat is homozygous for said disrupted Fel d I gene.

16. A cat according to claim 10, wherein said cat is fertile and capable of transmitting said disrupted Fel d I gene to its offspring.

17. A method for producing a transgenic cat comprising a disrupted Fel d I gene, comprising the steps of:
(a) introducing a cat stem cell comprising a disrupted Fel d I gene into a cat embryo;
(b) transplanting said embryo into a pseudopregnant cat; and
(c) allowing said cat embryo to mature into a cat.

18. A method according to claim 17, wherein said transgenic cat is heterozygous for said disrupted gene.

19. A method according to claim 17, wherein said cat is homozygous for said disrupted gene and wherein said cat does not produce the cat allergen Fel d I.

20. A method for producing a transgenic cat comprising a disrupted Fel d I gene, wherein said cat does not produce the cat allergen Fel d I, and wherein said cat is homozygous for said disrupted Fel d I gene, comprising the steps of:
(a) producing a first heterozygous transgenic cat according to claim 17;
(b) producing a second heterozygous transgenic cat according to claim 17, wherein said second cat is not the same sex as said first cat;
(c) breeding said first and second cats; and
(d) selecting transgenic cats which are homozygous for said disrupted Fel d I gene and do not produce Fel d I antigen.
